(19) United States
(12) Patent Application Publication
(10) Pub. No.: US 2026/0124280 A1
(43) Pub. Date: May 7, 2026

(54) SEMAGLUTIDE IN THE TREATMENT OF PERIPHERAL ARTERIAL DISEASE

(71) Applicant: Novo Nordisk A/S, Bagsvaerd (DK)

(72) Inventors: Andrei-Mircea Catarig, Herlev (DK); Diana Mathilde Roepcke, Vedbaek (DK); Anders Gaarsdal Holst, Gentofte (DK)

(21) Appl. No.: 19/378,410

(22) Filed: Nov. 4, 2025

Related U.S. Application Data
(60) Provisional application No. 63/716,349, filed on Nov. 5, 2024.

(30) Foreign Application Priority Data
Nov. 15, 2024 (EP) 24213241.3
Mar. 28, 2025 (EP) 25167042.8

(51) Int. Cl. A61K 38/26; A61P 9/10

(52) U.S. Cl. CPC A61K 38/26; A61P 9/10

(57) ABSTRACT

Disclosed herein is a further medical use of semaglutide, namely semaglutide in the treatment of intermittent claudication in a human subject with diabetes and peripheral arterial disease. Semaglutide is shown to have a positive impact on symptoms of intermittent claudication and therefore the functional limb capacity and quality of life of patients with type 2 diabetes and peripheral arterial disease.

### CROSS-REFERENCE TO RELATED APPLICATIONS

This application claims priority under 35 U.S.C. § 119 to U.S. Provisional Application 63/716,349, filed Nov. 5, 2024 and European Patent Applications 24213241.3, filed Nov. 15, 2024 and 25167042.8, filed Mar. 28, 2025; the contents of which are incorporated herein by reference.

The present invention relates to semaglutide in the improvement or treatment of intermittent claudication in a subject with peripheral arterial disease and diabetes.

### BACKGROUND

Peripheral arterial disease (PAD) affecting the lower extremities is a major cause of disability in older men and women, affecting more than 230 million people worldwide. The earliest, most common, and most disabling manifestation is functional decline and disability.

In PAD, atherosclerotic plaque builds up slowly on the inside of arteries. In the early stages of PAD, the arteries compensate for plaque buildup by dilating to preserve flow through the affected artery. Eventually, the artery cannot dilate any further, and the atherosclerotic plaque starts to narrow the arterial lumen.

Blood flow restriction caused by atherosclerosis represents the hallmark of PAD. The muscles of the lower extremity require increased blood flow during ambulation (walking), to meet their increased energy demand. When walking, patients with PAD reach a point at which collateral blood flow to affected tissues has been maximized and cannot compensate for the reduced perfusion to the lower extremity muscles caused by PAD. The supply-demand mismatch causes temporary ischemia of the muscles which manifests as pain, cramping or fatigue and ultimately makes the patient with PAD slow down or stop walking. Lowering the energy demands of the muscle (by slowing or stopping) allows the blood supply to “catch up,” and the ischemic symptoms resolve. The most characteristic ischemic symptoms of PAD—pain, cramping or fatigue in leg muscles which are brought on by walking and relieved with rest—are referred to as claudication.

Primarily when walking, symptoms occur in the muscle group distal to the artery that is narrowed or blocked. Patients with aortoiliac artery occlusive disease, therefore, have symptoms in the thigh and buttock muscles, whilst patients with femoropopliteal PAD have symptoms in their calf muscles. The walking distance at which symptoms occur depends on multiple factors, including disease severity, walking pace, terrain, and incline.

The cycle of blood flow restriction, increased energy demand and temporary muscle ischemia describes the pathophysiology of intermittent claudication due to PAD, which is stable or slowly progressive. PAD can, however, become progressively more severe, such that the blood flow cannot even meet the resting metabolic demands of the lower extremity. Non-healing wounds and ischemic ulcers manifest due to poor blood flow. In the most severe of cases, the toes or entire forefoot can become black and mummified as gangrene develops.

Risk factors include smoking, diabetes, obesity, high blood pressure, high cholesterol, increasing age, a family history of PAD, heart disease or stroke. Type 2 diabetes and smoking are the two leading causes of PAD, approximately one third (⅓) of all PAD patients having type 2 diabetes.

Current medical guidelines indicate that patients with symptomatic PAD should be treated with antiplatelet and antithrombotic therapy, cardiovascular risk reduction therapy (lipid lowering therapy, antihypertensive therapy and diabetes management), structured exercise and cilostazol (for chronic symptomatic PAD). Additionally, preventative foot care, influenza vaccination and SARS-CoV-2 vaccination are recommended.

Cilostazol, currently the only guideline-recommended medical therapy of PAD in the USA, is infrequently used due to side-effects that lead to treatment discontinuation in up to half of all treated patients, black box warnings for heart failure and a lack of other benefits.

Ultimately, PAD is associated with life and limb-threatening complications. Despite many advances in endovascular surgery, amputations of digits and limbs are not uncommon. Critical limb ischemia necessitating limb amputation is more common amongst PAD patients with T2D, compared to PAD patients without T2D.

Thus, there is a significant unmet medical need for a means of improving outcomes, such as walking ability, in patients with PAD.

### SUMMARY

Disclosed herein is a further medical use of semaglutide; namely, semaglutide for use in the treatment of intermittent claudication in a human subject with peripheral arterial disease. The subject may have diabetes, such as type 2 diabetes.

### DESCRIPTION

Semaglutide is a glucagon-like peptide 1 (GLP-1) receptor agonist and is the active pharmaceutical ingredient in Ozempic®, Rybelsus® and Wegovy®. Ozempic® is used
as an adjunct to diet and exercise to improve glycemic control in adults with type 2 diabetes mellitus;
to reduce the risk of major adverse cardiovascular events in adults with type 2 diabetes mellitus and established cardiovascular disease; and
to reduce the risk of sustained eGFR decline, end-stage kidney disease and cardiovascular death in adults with type 2 diabetes mellitus and chronic kidney disease.

Disclosed herein is a further medical use of semaglutide, namely semaglutide in the treatment of intermittent claudication in a human subject with peripheral arterial disease. The subject may be a human subject with peripheral arterial disease and diabetes. The subject may have type 1 diabetes. The subject may have type 2 diabetes. In a preferred embodiment, the subject has type 2 diabetes.

Surprisingly, semaglutide has a positive impact on the functional limb capacity and quality of life of subjects with intermittent claudication and peripheral arterial disease.

### Semaglutide

Semaglutide is also known by its chemical name: N-_{ε}²⁶-[2-(2-[2-(2-[2-(2-[4-(17-Carboxyheptadecanoylamino)-4(S)-carboxybutyrylamino]ethoxy)ethoxy]acetylamino)ethoxy]ethoxy)acetyl][Aib8,Arg34]GLP-1-(7-37)peptide. Semaglutide was first described in WO2006/097537 and subsequently in J. Med. Chem. 2015, 58, 18, 7370-7380. Semaglutide may be manufactured using methods well known to the person skilled in the art, such as that briefly described in WO2006/097537, Example 4.

Semaglutide may be present in the pharmaceutical formulation in its fully or partly ionised form; for example, one or more carboxylic acid groups (—COOH) may be deprotonated into the carboxylate group (—COO⁻) and/or one or more amino groups (—NH₂) may be protonated into the —NH₃⁺ group.

Semaglutide may be in the form of a salt, preferably a pharmaceutically acceptable salt.

### Peripheral Arterial Disease

In the context of the current invention, peripheral arterial disease (PAD) refers to disease and the ensuing partial occlusion of arteries that are distal to the aortoiliac junction, such as the common iliac artery, internal iliac artery, external iliac artery, femoral artery, popliteal artery, anterior tibial artery, posterior tibial artery or peroneal artery. As mentioned, above, PAD is predominantly caused by atherosclerosis.

### Diagnosis

Diagnostic testing for suspected PAD requires a multifaceted approach that may incorporate history and physical examination, ankle-brachial index (ABI), and additional physiological testing, as well as noninvasive and potentially invasive (angiography) imaging. Peripheral arterial disease may be diagnosed and classified using the ankle-brachial index (ABI), toe-brachial index (TBI), treadmill assessments and/or walking tests, as described below. Treadmill assessments and walking tests are standardised and are used to measure performance (walking capability).

### Ankle-Brachial Index (ABI) and Toe-Brachial Index (TBI)

Patients with peripheral arterial disease may have diminished or absent lower extremity pulses. This finding can be confirmed with the ankle-brachial index (ABI), an objective, bedside measure of lower extremity arterial perfusion. The ABI compares the systolic blood pressure at the ankle to the systolic pressure in the arm. The ABI is the highest systolic pressure measured at each ankle divided by the higher of the two systolic brachial pressures. A normal ABI typically range from 0.9 to 1.3-1.4. PAD is defined as having an ABI less than 0.9. Most patients with claudication have an ABI between 0.5 and 0.9. Patients with extremely low ABI's (less than 0.4) usually have ischemic rest pain or tissue loss. An ABI greater than 1.3-1.4 indicates arterial wall stiffening which can occur in patients with diabetes or renal failure. Patients with falsely elevated (greater than 1.3-1.4) ABIs require alternative imaging or physiologic studies to confirm the diagnosis of PAD. The toe-brachial index (TBI) can confirm the diagnosis.

In one study spanning 15 years, the average yearly ankle-brachial index (ABI) drop was 0.014 (J Vasc Surg 2001; 34:962; Eur Heart J Qual Care Clin Outcomes 2017; 3:208-15.)

### Treadmill Assessments

Treadmill assessments are used to determine a patients' so-called “maximum walking distance”, also referred to as the “absolute claudication distance”. Treadmill assessments are also used to determine a patients' so-called “pain-free walking distance”, also referred to as the “initial claudication distance”.

Treadmill assessments are only performed on treadmills meant for medical use.

The constant-load treadmill assessment is a standardised method for the functional assessment of patients with PAD. It is recommended in, for example, the European Medicines Agency (EMA) guidance on the clinical investigation of PAD.

The constant-load treadmill assessment referred to herein was performed at rate of 2 mph (3.2 km/h) and an inclination of 12%. The results of the constant-load test are expressed in units of distance walked.

Other standardised treadmill assessments known in the art can also be used to assess PAD patients' maximum walking distance and pain-free walking distance.

### Pain-Free Walking Distance on an Inclined Treadmill

In the constant-load treadmill test, the pain-free distance walked is the distance that has been walked by the time pain starts to be felt in either leg.

### Maximum Walking Distance on an Inclined Treadmill

In the constant-load treadmill test, and after having indicated when pain is first felt, the patient continues walking on the treadmill for as long as possible until pain limited further activity. This distance is noted as being the maximum walking distance.

### Pain-Free Walking Distance on a Flat Treadmill

Proper classification of Fontaine Ila claudication can be confirmed on a flat treadmill, mimicking a normal walking situation. If a patient is able to walk at least 200 m (656 feet) on a flat treadmill with a fixed speed of 3.2 km/h (2 mph) before onset of pain in either leg, the patient has Fontaine Ila claudication.

### Six Minutes Walking Test

In the six minute walking test, patients walk back and forth along a 100-foot long corridor and the distance walked at 6 minutes is recorded. Pauses and change in speed are allowed.

It has previously been reported that, amongst people with PAD, approximately 8 and 20 meter improvements in the 6-minute distance walked represented small and large improvements, respectively, in the walking ability of adults with PAD (J. Vasc. Surg. 2021; 73:267-76).

### Classification

The Fontaine classification system (Hel Chir Acta. 1954; 21(5-6):499-533) grades the clinical presentation of PAD into four stages, as shown in Table 1. Walking ability is measured on flat terrain for the purpose of grading patients according to the Fontaine classification.

The Fontaine classification system is typically used for clinical research.

TABLE 1
Fontaine classification

| Grade | Symptoms |
|---|---|
| Stage I | Asymptomatic, incomplete blood vessel obstruction |
| Stage II | Mild claudication pain in limb |
| Stage IIA | Claudication at a distance >200 m |
| Stage IIB | Claudication at a distance <200 m |
| Stage III | Rest pain, mostly in the feet |
| Stage IV | Necrosis and/or gangrene of the limb |

Fontaine's classification was later adapted by Rutherford (J Vasc Surg. 1997; 26(3):517-538), as shown in Table 2. The Rutherford Classification system is more commonly used in clinical practice.

Participants of the trial described in Example 1 had Fontaine stage IIA PAD at the start of treatment, corresponding to Rutherford classification grade 1, categories 1 and 2 (mild and moderate claudication).

TABLE 2
Rutherford classification

| Grade | Category | Clinical description |
|---|---|---|
| 0 | 0 | Asymptomatic-no hemodynamically significant |
| | | occlusive disease |
| I | 1 | Mild claudication |
| | 2 | Moderate claudication |
| | 3 | Severe claudication |
| II | 4 | Ischemic rest pain |
| III | 5 | Minor tissue loss - nonhealing ulcer, focal gangrene |
| | | with diffuse pedal ischemia |
| | 6 | Major tissue loss-extending above TM level, |
| | | functional foot no longer salvageable |

In one study spanning 15 years, the mean self-reported walking distance was reduced by 8.4 metres per year (J Vasc Surg 2001; 34:962; Eur Heart J Qual Care Clin Outcomes 2017; 3:208-15).

### Medical Treatment

Semaglutide may be administered by parenteral injection, such as by subcutaneous injection.

Semaglutide may be orally administered.

The term “treatment”, as used herein, refers to the medical therapy of a human subject in need thereof. Said subject is expected to have undergone physical examination by a medical practitioner, who has given a tentative or definitive diagnosis which would indicate that the use of said specific treatment is beneficial to the health of the patient. The timing of the treatment may vary from one individual to another, according to the status quo of the subject's health. Thus, said treatment may be symptomatic and/or curative. Said treatment may result in an improvement of the subject's physical health. Said treatment may result in an improvement of the subject's physical ability. Said treatment may indirectly result in an improvement in the subject's mental health.

The pharmaceutical formulation disclosed herein may be administered to a human subject, such as a human adult subject.

Semaglutide may be the only active ingredient administered in the method of the present invention. Semaglutide may be the sole GLP-1 receptor agonist administered in the method of the present invention.

Semaglutide may be administered together with one or more additional active ingredients, which are not GLP-1 receptor agonists, in the method of the present invention.

### Pharmaceutical Formulations

Semaglutide may be administered in a pharmaceutical formulation.

The terms “pharmaceutical formulation”, “formulation”, “pharmaceutical composition” and “composition” are used interchangeably herein and refer to pharmaceutical formulations suitable for administration to a subject in need thereof. To the person skilled in the art, pharmaceutical formulations and compositions are also known as “drug products”.

The pharmaceutical formulation may be a liquid formulation, such as an aqueous formulation, comprising semaglutide. The liquid formulation may have a pH of about 5.5-8.0, such as about 5.6-7.8, such as about 5.6-7.6. The liquid formulation may have a pH of about 7.2-7.6, such as about 7.4. Where the pharmaceutical formulation is a liquid formulation, the formulation may comprise 0.1-10 mg/ml semaglutide.

The pharmaceutical formulation may alternatively be a solid formulation, such as a tablet, comprising semaglutide. Said solid formulation for oral administration may include an absorption enhancer, such as salcaprozic acid, or a salt thereof, such as salcaprozate sodium, also known as sodium N-(8[2-hydroxybenzoyl]-amino)caprylate (SNAC). The solid formulation for oral administration may comprise 3 mg, 7 mg or 14 mg semaglutide. The solid formulation for oral administration may comprise 1.5 mg, 4.0 mg, 9 mg, 25 mg or 50 mg semaglutide. The formulation may comprise one or more pharmaceutically acceptable excipients.

pH values referred to herein may be measured at room temperature.

### Medical Devices

A medical device may be used for the administration of a pharmaceutical formulation which is a liquid formulation comprising semaglutide. A medical device may thus be used for the administration of a predetermined dose of semaglutide. A medical device may be used for the administration, in a single injection, of a single dose of semaglutide. The medical device may be a syringe device, such as pen injector device.

### Doses

An effective amount of semaglutide may be administered to the subject in need thereof.

The dose may be administered approximately once daily. The dose may be administered in a solid composition, such as in a tablet for peroral administration, approximately once daily. Said dose may be taken on an empty stomach. The subject preferably takes said dose in said tablet after a period of fasting of at least 8 hours' duration. The subject preferably does not eat until at least 30 minutes after having taken the tablet.

The dose may be administered in an injectable pharmaceutical formulation approximately once weekly. The dose may be administered once weekly at any time of day, with or without meals. One interval between two fixed doses may be about 2 days, about 3 days, about 4 days, about 5 days, about 6 days, about 7 days, about 8 days, about 9 days or about 10 days.

In a preferred embodiment, a dose is administered once weekly. In a preferred embodiment, a fixed maintenance dose is administered approximately once weekly. In a preferred embodiment, a fixed maintenance dose is administered approximately once every 7 days.

The term “once weekly” administration refers to administration once every seven days. The term “about once weekly” or “approximately once weekly”, as used herein, refers to administration more or less than once every seven days and includes situations where subject compliance can be imperfect; and/or that a dose can be missed; and/or that the subject needs or wishes to change the day of the week of administration. The interval between two fixed doses may thence be about 2 days, about 3 days, about 4 days, about 5 days, about 6 days, about 7 days, about 8 days, about 9 days or about 10 days.

If a dose is missed, the dose is preferably administered within 5 days of the missed dose. If more than 5 days have passed, the missed dose is preferably skipped, and the next dose administered on the regularly scheduled day. In each case, the subject can then resume his or her regular once weekly dosing schedule.

If necessary or desired, the day of weekly administration can be changed, in which case “once weekly” administration encompasses, as an exception, the time between two doses being at least 2 days (>48 hours).

Upon initiation of treatment, it may be beneficial to administer ascending doses of semaglutide to the subject in need thereof. Once the subject is acclimatised to the treatment, it may be beneficial to administer maintenance doses of semaglutide to the subject in need thereof. Treatment may be once weekly, with dose escalation approximately once every four weeks until the maintenance dose is achieved.

The dose of semaglutide administered may be 0.25-50 mg.

When administered in the form of a liquid formulation for parenteral administration (that is, a solution for injection), such as for subcutaneous administration, the dose of semaglutide administered may be 0.2-20 mg. The dose of semaglutide administered may be about 0.25-16 mg, such as about 0.25-9.0 mg, such as about 0.25-4.5 mg, such as about 0.25-2.4 mg.

The dose of semaglutide administered may be about 0.25 mg.

At the start of treatment, the dose of semaglutide administered may be about 0.25 mg. At the start of treatment, the dose of semaglutide administered may be about 0.25 mg once weekly. At the start of treatment, the dose of semaglutide administered may be about 0.25 mg approximately once weekly.

After 4 weeks on the 0.25 mg dosage, the dose of semaglutide administered may be increased to 0.5 mg once weekly. After 4 weeks on the 0.25 mg dosage, the dose of semaglutide administered may be increased to 0.5 mg approximately once weekly.

The dose of semaglutide administered may be about 0.5 mg.

After at least 4 weeks on the 0.5 mg dose and if additional glycemic control is needed, the dose of semaglutide administered may be increased to 1 mg once weekly. After at least 4 weeks on the 0.5 mg dose and if additional glycemic control is needed, the dose of semaglutide administered may be increased to 1 mg approximately once weekly.

To reduce the risk of sustained eGFR decline, end-stage kidney disease and cardiovascular death, the dosage may be increased to 1 mg once weekly after at least 4 weeks on the 0.5 mg dosage. To reduce the risk of sustained eGFR decline, end-stage kidney disease and cardiovascular death, the dosage may be increased to 1 mg approximately once weekly after at least 4 weeks on the 0.5 mg dosage.

The dose of semaglutide administered may be about 1.0 mg.

If additional glycemic control is needed, the dosage may be increased to 2 mg once weekly after at least 4 weeks on the 1 mg dosage. If additional glycemic control is needed, the dosage may be increased to 2 mg approximately once weekly after at least 4 weeks on the 1 mg dosage.

The dose of semaglutide administered may be about 1.7 mg.

The dose of semaglutide administered may be about 2.4 mg.

The dose of semaglutide administered may be about 3.6 mg.

The dose of semaglutide administered may be about 4.5 mg.

The dose of semaglutide administered may be about 4.8 mg.

The dose of semaglutide administered may be about 6.0 mg.

The dose of semaglutide administered may be about 6.9 mg.

The dose of semaglutide administered may be about 7.2 mg.

The dose of semaglutide administered may be about 8.0 mg.

The dose of semaglutide administered may be about 9.0 mg.

The dose of semaglutide administered may be about 12 mg.

The dose of semaglutide administered may be about 16.0 mg.

The dose of semaglutide administered may be 0.25 mg once-weekly for four weeks and 0.5 mg once-weekly thereafter. The dose of semaglutide administered may be 0.25 mg approximately once-weekly for four weeks and 0.5 mg approximately once-weekly thereafter.

The dose of semaglutide administered may be 0.25 mg once-weekly for four weeks, 0.5 mg once-weekly for four weeks and 1.0 mg once-weekly thereafter. The dose of semaglutide administered may be 0.25 mg approximately once-weekly for four weeks, 0.5 mg approximately once-weekly for four weeks and 1.0 mg approximately once-weekly thereafter.

When administered in the form of a solid formulation for oral administration, such as a tablet, the dose of semaglutide administered may be 1-50 mg.

The dose of semaglutide administered may be 1.5 mg.

The dose of semaglutide administered may be 1.5 mg once daily. The dose of semaglutide administered may be 1.5 mg once daily for one month, after which the dose may be increased. The dose of semaglutide administered may be increased to 4 mg once daily.

The dose of semaglutide administered may be 3 mg.

The dose of semaglutide administered may be 3 mg once daily. The dose of semaglutide administered may be 3 mg once daily for one month, after which the dose may be increased. The dose of semaglutide administered may be increased to 7 mg once daily.

The dose of semaglutide administered may be 4 mg.

The dose of semaglutide administered may be 4 mg once daily. The dose of semaglutide administered may be 4 mg once daily for one month, after which the dose may be increased. The dose of semaglutide administered may be increased to 9 mg once daily.

The dose of semaglutide administered may be 7 mg.

The dose of semaglutide administered may be 7 mg once daily. The dose of semaglutide administered may be 7 mg once daily for one month, after which the dose may be increased. The dose of semaglutide administered may be increased to 14 mg once daily.

The dose of semaglutide administered may be 9 mg.

The dose of semaglutide administered thereafter may be 9 mg once daily. The dose of semaglutide administered may be 9 mg once daily for one month, after which the dose may be increased. The dose of semaglutide administered may be increased to 25 mg once daily.

The dose of semaglutide administered may be 14 mg.

The dose of semaglutide administered may be 14 mg once daily.

The dose of semaglutide administered may be 25 mg.

The dose of semaglutide administered thereafter may be 25 mg once daily. The dose of semaglutide administered thereafter may be 25 mg once daily for one month, after which the dose may be increased. The dose of semaglutide administered may be increased to 50 mg once daily.

The dose of semaglutide administered may be 50 mg.

The dose of semaglutide administered may be 50 mg once daily.

Herein, specific values given in relation to numbers or intervals may be construed as being the specific value or the approximate value; such as plus or minus 0.49, when pH is measured. Unless otherwise stated, ranges herein include their end points. In some embodiments the term “a” means “one or more”. Unless otherwise indicated herein, terms presented in singular form may also include the plural situation. Herein the term “about” means±10% of the value referred to and includes the value unless otherwise specified. In some embodiments the term “comprise” as used herein includes the term “consist of”.

Following is a non-limiting list of embodiments of the present invention.

### Embodiments

1. Semaglutide for use in the treatment of peripheral arterial disease (PAD).
2. Semaglutide for use in the treatment of symptomatic peripheral arterial disease (PAD).
3. Semaglutide for use in reducing the symptoms of peripheral arterial disease (PAD).
4. Semaglutide for use in the treatment or improvement of intermittent claudication in a human subject with peripheral arterial disease (PAD) with intermittent claudication.
5. Semaglutide for use in the treatment or improvement of intermittent claudication in a human subject with diabetes and peripheral arterial disease (PAD).
6. Semaglutide for use in the treatment or improvement of intermittent claudication in a human subject with type 1 or type 2 diabetes and peripheral arterial disease (PAD).
7. Semaglutide for use in the treatment or improvement of intermittent claudication in a human subject with type 2 diabetes and peripheral arterial disease (PAD).
8. Semaglutide for use according to embodiment 7 and as an adjunct to diet and exercise to improve glycemic control in an adult human with type 2 diabetes mellitus.
9. Semaglutide for use according to embodiment 7 or 8 and to reduce the risk of major adverse cardiovascular events in adults with type 2 diabetes mellitus and established cardiovascular disease.
10. Semaglutide for according to any one of embodiments 7-9 and to reduce the risk of sustained eGFR decline, end-stage kidney disease and cardiovascular death in adults with type 2 diabetes mellitus and chronic kidney disease.
11. Semaglutide for use according to any one of embodiments 1-10, wherein said subject has intermittent claudication corresponding to Fontaine Stage IIA.
12. Semaglutide for use according to any one of embodiments 1-11, wherein said treatment increases the maximum distance that said subject is capable of walking.
13. Semaglutide for use according to embodiment 12, wherein the maximum distance that the subject is capable of walking is at least 600 meters at the start of treatment.
14. Semaglutide for use according to embodiment 13, wherein the maximum distance that the subject is capable of walking is measured on a treadmill set at a speed of 3.2 km/hour and at a 12% incline.
15. Semaglutide for use according to any one of embodiments 1-14, wherein said treatment increases the distance that said subject is capable of walking without feeling pain.
16. Semaglutide for use according to embodiment 15, wherein the distance that said subject is capable of walking without feeling pain is at least 200 m at the start of treatment.
17. Semaglutide for use according to embodiment 16, wherein the distance that the subject is capable of walking, pain-free, is measured on a treadmill set at a speed of 3.2 km/hour and at a 12% incline.
18. Semaglutide for use according to any one of embodiments 1-17, wherein said human subject has an ankle-brachial index (ABI) which is equal to or less than 0.90 and/or a toe-brachial index (TBI) which is equal to or less than 0.70 at the start of treatment.
19. Semaglutide for use according to embodiment 18, wherein said human subject has an ankle-brachial index (ABI) between 0.4 and 0.9 and/or a toe-brachial index (TBI) equal to or less than 0.70 at the start of treatment.
20. Semaglutide for use according to embodiment 18, wherein said human subject has an ankle-brachial index (ABI) which is equal to or less than 0.90 at the start of treatment.
21. Semaglutide for use according to embodiment 18, wherein said human subject has an ankle-brachial index (ABI) which is equal to or more than 0.4 at the start of treatment.
22. Semaglutide for use according to embodiment 18, wherein said human subject has an ankle-brachial index between 0.4 and 0.9 at the start of treatment.
23. Semaglutide for use according to any one of embodiments 1-22, wherein said treatment increases the ankle-brachial-index (ABI) of said subject.
24. Semaglutide for use according to embodiment 23, wherein said treatment increases the ankle-brachial-index (ABI) of the subject by at least 5%.
25. Semaglutide for use according to embodiment 19, wherein said human subject has a toe-brachial index (TBI) equal to or less than 0.70 at the start of treatment.
26. Semaglutide for use according to any one of embodiments 1-25, wherein said treatment increases the toe-brachial-index (TBI) of said subject.
27. A method of treating and/or improving intermittent claudication in a human subject with peripheral arterial disease, comprising administering semaglutide to a subject in need thereof.
28. A method of treating and/or improving intermittent claudication in a human subject with diabetes and peripheral arterial disease, comprising administering semaglutide to a subject in need thereof.
29. A method of treating and/or improving intermittent claudication in a human subject with type 1 or type 2 diabetes and peripheral arterial disease, comprising administering semaglutide to a subject in need thereof.
30. A method of treating and/or improving intermittent claudication in a human subject with type 2 diabetes and peripheral arterial disease, comprising administering semaglutide to a subject in need thereof.
31. The method according to any one of embodiments 27-30, wherein said subject has intermittent claudication corresponding to Fontaine stage IIA at the start of treatment.
32. The method according to any one of embodiments 27-31, wherein the improvement of intermittent claudication comprises an increase in the maximum distance capable of being walked by the subject.
33. The method according to embodiment 32, wherein the maximum distance that the subject is capable of walking is at least 600 meters at the start of treatment.
34. The method according to embodiment 32, wherein the maximum distance capable of being walked by the subject is measured on a treadmill set at a speed of 3.2 km/hour and at a 12% incline.
35. The method according to embodiment 32, wherein the administration of semaglutide produces a 10% improvement, or more, in maximum walking distance.
36. The method according to embodiment 32, wherein the administration of semaglutide produces a 13% improvement in maximum walking distance.
37. The method according to any one of embodiments 1-36, wherein the improvement of intermittent claudication comprises an increase in the distance that the subject is capable of walking without feeling pain.
38. The method according to embodiment 37, wherein the distance that said subject is capable of walking without feeling pain is at least 200 m at the start of treatment.
39. The method according to embodiment 37, wherein the distance that the subject is capable of walking without feeling pain is measured on a treadmill set at a speed of 3.2 km/hour and at a 12% incline.
40. The method according to embodiment 37, wherein the improvement of intermittent claudication comprises an improvement of 5%, or more, in the distance that the subject is capable of walking without feeling pain.
41. The method according to embodiment 37, wherein the improvement of intermittent claudication comprises an improvement of 8% in the distance that the subject is capable of walking without feeling pain.
42. The method according to any one of embodiments 1-41, wherein the treating and/or improving of intermittent claudication comprises an increase in the ankle-brachial index (ABI) and/or the toe-brachial index (TBI) of said human subject.
43. The method according to any one of embodiments 1-42, wherein said human subject has an ankle-brachial index (ABI) which is equal to or less than 0.90 or a toe-brachial index (TBI) which is equal to or less than 0.70 at the start of treatment.
44. The method according to embodiment 1-43, wherein the subject has an ankle-brachial-index (ABI) equal to or less than 0.90 at the start of treatment.
45. The method according to embodiment 1-44, wherein the subject has an ankle-brachial-index (ABI) equal to or more than 0.40 at the start of treatment.
46. The method according to embodiment 1-45, wherein the subject has an ankle-brachial index (ABI) between 0.4 and 0.9 at the start of treatment.
47. The method according to any one of embodiments 42-46, wherein administration of semaglutide produces an increase in the ankle-brachial index (ABI) of the subject.
48. The method according to embodiment 47, wherein the administration of semaglutide produces a 5% improvement, or more, in the ankle-brachial-index (ABI) of the subject.
49. The method according to any one of embodiments 42-46, wherein the treating or improving of intermittent claudication comprises an increase in the toe-brachial index (TBI) of the subject.
50. The method according to embodiment 49, wherein said subject has a toe-brachial index (TBI) equal to or less than 0.70 at the start of treatment.
51. Use of semaglutide in the preparation of a medicament for the treatment and/or improvement of intermittent claudication in a human subject with peripheral arterial disease (PAD).
52. Use of a formulation in the preparation of a medicament for the treatment and/or improvement of intermittent claudication in a human subject with peripheral arterial disease (PAD), wherein said formulation comprises semaglutide and one or more pharmaceutically acceptable excipients.
53. Use of semaglutide in the preparation of a medicament for the treatment and/or improvement of intermittent claudication in a human subject with peripheral arterial disease (PAD) and diabetes.
54. Use of semaglutide in the preparation of a medicament for the treatment and/or improvement of intermittent claudication in a human subject with peripheral arterial disease (PAD) and type 1 or 2 diabetes.
55. Use of a formulation in the preparation of a medicament for the treatment and/or improvement of intermittent claudication in a subject with peripheral arterial disease (PAD) with and diabetes, wherein said formulation comprises semaglutide and one or more pharmaceutically acceptable excipients.
56. Use of a formulation in the preparation of a medicament for the treatment and/or improvement of intermittent claudication in a subject with peripheral arterial disease (PAD) and type 1 or 2 diabetes, wherein said formulation comprises semaglutide and one or more pharmaceutically acceptable excipients.
57. Use of semaglutide in the preparation of a medicament for the treatment and/or improvement of intermittent claudication in a human subject with peripheral arterial disease (PAD) and type 2 diabetes.
58. Use of a formulation in the preparation of a medicament for the treatment and/or improvement of intermittent claudication in a subject with peripheral arterial disease (PAD) and type 2 diabetes, wherein said formulation comprises semaglutide and one or more pharmaceutically acceptable excipients.
59. Use according to any one of embodiments 51-58, wherein said subject has Fontaine Stage IIA intermittent claudication.
60. Use according to any one of embodiments 51-59, wherein said treatment increases the maximum distance that said subject is capable of walking.
61. Use according to embodiment 60, wherein the maximum distance that the subject is capable of walking is at least 600 meters at the start of treatment.
62. Use according to embodiment 60, wherein the maximum distance that said subject is capable of walking is measured on a treadmill set at a speed of 3.2 km/hour and at a 12% incline.
63. Use according to any one of embodiments 51-60, wherein the distance that said subject is capable of walking without feeling pain is at least 200 m at the start of treatment.
64. Use according to embodiment 63, wherein said treatment increases the distance that said subject is capable of walking without feeling pain.
65. Use according to embodiment 63, wherein the distance that the subject is capable of walking without feeling pain is measured on a treadmill set at a speed of 3.2 km/hour and at a 12% incline.
66. Use according to any one of embodiments 51-65, wherein said subject has an ankle-brachial index (ABI) which is equal to or less than 0.90 and/or a toe-brachial index (TBI) which is equal to or less than 0.70 at the start of treatment.
67. Use according to embodiment 66, wherein said subject has an ankle-brachial index (ABI) between 0.4 and 0.9 and/or a toe-brachial index (TBI) equal to or less than 0.70 at the start of treatment.
68. Use according to embodiment 66, wherein said subject has an ankle-brachial index (ABI) equal to or less than 0.9 at the start of treatment.
69. Use according to embodiment 66, wherein said subject has an ankle-brachial index (ABI) equal to or more than 0.4 at the start of treatment.
70. Use according to embodiment 66, wherein said subject has an ankle-brachial index (ABI) between 0.4 and 0.9 at the start of treatment.
71. Use according to any one of embodiments 66-70, wherein said treatment increases the ankle-brachial-index (ABI) of said subject.
72. Use according to embodiment 71, wherein said treatment increases the ankle-brachial-index (ABI) of the subject by 5% or more.
73. Use according to embodiment 66, wherein said subject has a toe-brachial index (TBI) equal to or less than 0.70 at the start of treatment.
74. Use according to any one of embodiments 51-73, wherein said treatment increases the toe-brachial-index (TBI) of said subject.
75. Use according to any one of embodiments 51-74, wherein said treatment increases the ankle-brachial index (ABI) and/or the toe-brachial index (TBI) of said human subject.
76. Semaglutide for use according to any one of embodiments 1-26 or 51-75, wherein semaglutide is administered once daily, or approximately once daily.
77. Semaglutide for use according to any one of embodiments 1-26 or 51-75, wherein semaglutide is administered once daily, or approximately once daily, in a therapeutically effective amount.
78. Semaglutide for use according to any one of embodiments 1-26 or 51-75, wherein semaglutide is administered once weekly, or approximately once weekly.
79. Semaglutide for use according to any one of embodiments 1-26 or 51-75, wherein semaglutide is administered once weekly, or approximately once weekly, in a therapeutically effective amount.
80. Semaglutide for use according to any one of embodiments 1-26 or 51-75, wherein semaglutide is administered in an amount of 0.2-50 mg.
81. Semaglutide for use according to any one of embodiments 1-26 or 51-75, wherein semaglutide is administered in the form of a solid composition, such as a tablet.
82. Semaglutide for use according to any one of embodiments 1-26 or 51-75, wherein said semaglutide is administered in a dose of 1-50 mg.
83. Semaglutide for use according to any one of embodiments 1-26 or 51-75, wherein semaglutide is administered in the form of a liquid formulation for injection.
84. Semaglutide for use according to embodiment 83, wherein said semaglutide is administered once weekly, or approximately once weekly.
85. Semaglutide for use according to embodiment 83, wherein said semaglutide is administered in a dose of up to 10 mg.
86. Semaglutide for use according to embodiment 83, wherein said semaglutide is administered in an amount of of 0.2-10 mg.
87. Semaglutide for use according to embodiment 83, wherein semaglutide is administered in an amount of 0.25-2.4 mg.
88. Semaglutide for use according to embodiment 83, wherein semaglutide is administered once weekly in an amount of 0.25-2.4 mg.
89. Semaglutide for use according to embodiment 83, wherein semaglutide is administered in an amount of 0.25-1.0 mg.
90. Semaglutide for use according to embodiment 83, wherein semaglutide is administered once weekly in an amount of 0.25-1.0 mg.
91. Semaglutide for use according to embodiment 83, wherein semaglutide is administered in an amount of about 0.25 mg.
92. Semaglutide for use according to embodiment 83, wherein semaglutide is administered once weekly in an amount of about 0.25 mg.
93. Semaglutide for use according to embodiment 83, wherein semaglutide is administered in an amount of about 0.5 mg.
94. Semaglutide for use according to embodiment 83, wherein semaglutide is administered once weekly in an amount of about 0.5 mg.
95. Semaglutide for use according to embodiment 83, wherein semaglutide is administered in an amount of about 1.0 mg.
96. Semaglutide for use according to embodiment 83, wherein semaglutide is administered once weekly in an amount of about 1.0 mg.
97. Semaglutide for use according to embodiment 83, wherein semaglutide is administered in an amount of about 1.7 mg.
98. Semaglutide for use according to embodiment 83, wherein semaglutide is administered once weekly in an amount of about 1.7 mg.
99. Semaglutide for use according to embodiment 83, wherein semaglutide is administered in an amount of about 2.0 mg.
100. Semaglutide for use according to embodiment 83, wherein semaglutide is administered once weekly in an amount of about 2.0 mg.
101. Semaglutide for use according to embodiment 83, wherein semaglutide is administered in an amount of about 2.4 mg.
102. Semaglutide for use according to embodiment 83, wherein semaglutide is administered once weekly in an amount of about 2.4 mg.
103. Semaglutide for use according to embodiment 83, wherein semaglutide is administered in an amount of about 7.2 mg.
104. Semaglutide for use according to embodiment 83, wherein semaglutide is administered once weekly in an amount of about 7.2 mg.
105. Semaglutide for use according to any one of embodiments 1-26 or 51-75, wherein semaglutide is administered in the form of a solid formulation for oral administration.
106. Semaglutide for use according to embodiment 105, wherein said semaglutide is administered once daily, or approximately once daily.
107. Semaglutide for use according to embodiment 105, wherein semaglutide is administered in an amount of about 1.5 mg.
108. Semaglutide for use according to embodiment 105, wherein semaglutide is administered in an amount of 1.5 mg.
109. Semaglutide for use according to embodiment 105, wherein semaglutide is administered in an amount of about 3.0 mg.
110. Semaglutide for use according to embodiment 105, wherein semaglutide is administered in an amount of 3.0 mg.
111. Semaglutide for use according to embodiment 105, wherein semaglutide is administered in an amount of about 4.0 mg.
112. Semaglutide for use according to embodiment 105, wherein semaglutide is administered in an amount of 4.0 mg.
113. Semaglutide for use according to embodiment 105, wherein semaglutide is administered in an amount of about 7.0 mg.
114. Semaglutide for use according to embodiment 105, wherein semaglutide is administered in an amount of 7.0 mg.
115. Semaglutide for use according to embodiment 105, wherein semaglutide is administered in an amount of about 9.0 mg.
116. Semaglutide for use according to embodiment 105, wherein semaglutide is administered in an amount of 9.0 mg.
117. Semaglutide for use according to embodiment 105, wherein semaglutide is administered in an amount of about 14 mg.
118. Semaglutide for use according to embodiment 105, wherein semaglutide is administered in an amount of about 25 mg.
119. Semaglutide for use according to embodiment 105, wherein semaglutide is administered in an amount of about 50 mg.
120. Semaglutide for use according to any one of embodiments 105-106, wherein semaglutide is administered in an amount of 3 mg, 7 mg or 14 mg.
121. Semaglutide for use according to any one of embodiments 105-106, wherein semaglutide is administered in an amount of 1.5 mg, 4.0 mg, 9 mg, 25 mg or 50 mg.
122. Semaglutide for use according to any one of embodiments 1-26, 51-75, 78-80, 82-104, wherein said semaglutide is administered parenterally.
123. Semaglutide for use according to embodiment 122, wherein said semaglutide is administered subcutaneously.
124. Semaglutide for use according to any one of embodiments 122-123, wherein said semaglutide is administered in the form of a liquid formulation.
125. Semaglutide for use according to any one of embodiments 105-106, wherein said semaglutide is administered in the form of a tablet.
126. The method according to any one of embodiments 27-50, wherein semaglutide is administered once daily, or approximately once daily.
127. The method according to any one of embodiments 27-50, wherein semaglutide is administered once weekly, or approximately once weekly.
128. The method according to any one of embodiments 27-50 or 126, wherein semaglutide is administered once daily, or approximately once daily, in a therapeutically effective amount.
129. The method according to any one of embodiments 27-50, wherein semaglutide is administered once weekly, or approximately once weekly, in a therapeutically effective amount.
130. The method according to any one of embodiments 27-50 or 126-129, wherein semaglutide is administered in an amount of 0.2-50 mg.
131. The method according to any one of embodiments 27-50, 126, 128 or 130, wherein semaglutide is administered in the form of a solid composition, such as a tablet.
132. The method according to any one of embodiments 27-50, 126, 129-130, wherein said semaglutide is administered in a dose of 1-50 mg.
133. The method according to any one of embodiments 27-50, 127, 129, 130 or 132, wherein semaglutide is administered in the form of a liquid formulation for injection.
134. The method according to any one of embodiments 27-50, 126-133, wherein said semaglutide is administered in a dose of up to 10 mg.
135. The method according to any one of embodiments 27-50, 126-134, wherein said semaglutide is administered in a dose of 0.2-10 mg.
136. The method according to any one of embodiments 27-50, 126-127, 129-130, 132-135, wherein semaglutide is administered once weekly in an amount of 0.25-2.4 mg.
137. The method according to any one of embodiments 27-50, 126, 128, 130, 132-136, wherein semaglutide is administered once weekly in an amount of 0.25-1.0 mg.
138. The method according to any one of embodiments 27-50, 126, 128, 130, 132-137, wherein semaglutide is administered once weekly in an amount of about 0.25 mg.
139. The method according to any one of embodiments 27-50, 126, 128, 130, 132-138, wherein semaglutide is administered once weekly in an amount of about 0.5 mg.
140. The method according to any one of embodiments 27-50, 126, 128, 130, 132-139, wherein semaglutide is administered once weekly in an amount of about 1.0 mg.
141. The method according to any one of embodiments 27-50, 126, 128, 131-132, 134-135, wherein semaglutide is administered in an amount of about 1.5 mg.
142. The method according to any one of embodiments 27-50, 127, 129, 130, 132-136, wherein semaglutide is administered in an amount of about 1.7 mg.
143. The method according to any one of embodiments 27-50, 127, 129, 130, 132-136, wherein semaglutide is administered in an amount of about 2.0 mg.
144. The method according to any one of embodiments 27-50, 127, 129, 130, 132-136, wherein semaglutide is administered in an amount of about 2.4 mg.
145. The method according to any one of embodiments 27-50, 126, 128, 131-132, 134-135, wherein semaglutide is administered in an amount of about 3.0 mg.
146. The method according to any one of embodiments 27-50, 126, 128, 131-132, 134-135, wherein semaglutide is administered in an amount of about 4.0 mg.
147. The method according to any one of embodiments 27-50, 126, 128, 131-132, 134-135, wherein semaglutide is administered in an amount of about 7.0 mg.
148. The method according to any one of embodiments 27-50, 127, 129, 130, 132-136, wherein semaglutide is administered in an amount of about 7.2 mg.
149. The method according to any one of embodiments 27-50, 126, 128, 131-132, 134-135, wherein semaglutide is administered in an amount of about 9.0 mg.
150. The method according to any one of embodiments 27-50, 126, 128, 131-132, 134-135, wherein semaglutide is administered in an amount of about 14 mg.
151. The method according to any one of embodiments 27-50, 126, 128, 131-132, 134-135, wherein semaglutide is administered in an amount of about 25 mg.
152. The method according to any one of embodiments 27-50, 126, 128, 131-132, 134-135, wherein semaglutide is administered in an amount of about 50 mg.
153. The method according to any one of embodiments 27-50, 127, 129, 130, 132-136, wherein said semaglutide is administered parenterally.
154. The method according to any one of embodiments 27-50, 127, 129, 130, 132-136 or 153, wherein said semaglutide is administered subcutaneously.
155. The method according to any one of embodiments 27-50, 127, 129, 130, 132-136, 153-154, wherein said semaglutide is administered in the form of a liquid formulation.
156. The method according to any one of embodiments 27-50, 126, 128, 131-132, 134-135, 145-147, 149-152, wherein said semaglutide is administered orally.
157. The method according to any one of embodiments 27-50, 126, 128, 131-132, 134-135, 145-147, 149-152, wherein said semaglutide is administered in the form of a solid.
158. The method according to any one of embodiments 27-50, 126, 128, 131-132, 134-135, 145-147, 149-152, wherein said semaglutide is administered in the form of a tablet.
159. A pharmaceutical formulation comprising semaglutide for use according to any one of embodiments 1-26 or 51-75, or for use in the method according to any one of embodiments 27-50.
160. The pharmaceutical formulation according to embodiment 159, wherein the formulation is a liquid formulation.
161. The liquid formulation according to embodiment 159, comprising 0.25-22 mg/ml semaglutide.
162. The liquid formulation according to embodiment 159, comprising 0.25-10 mg/ml semaglutide.
163. The pharmaceutical formulation according to any one of embodiments 159-162, wherein the formulation has a pH of 5.5-8.0, such as a pH of 5.6-7.6.
164. The pharmaceutical formulation according to embodiment 159, wherein the formulation is a solid formulation.
165. The pharmaceutical formulation according to embodiment 159, wherein the formulation is a tablet.
166. The tablet according to embodiment 159, comprising about 1-50 mg semaglutide.

### EXAMPLES

### Example 1: Functional Capacity in Human Adults with Type 2 Diabetes (T2D) and Peripheral Arterial Disease (PAD)

STRIDE was a 52-week, randomised, double-blind, placebo-controlled trial comparing 1 mg subcutaneously administered semaglutide with placebo, added to standard-of-care and administered once-weekly in adult patients with type 2 diabetes (T2D) and peripheral arterial disease (PAD) with intermittent claudication.

The primary objective was to demonstrate the effect of the once-weekly, subcutaneous administration of 1 mg semaglutide on walking ability (change in maximum walking distance from baseline to week 52, compared with placebo) in patients with type 2 diabetes and peripheral arterial disease with intermittent claudication. The primary endpoint was a change in maximum walking distance on a constant load treadmill test (with a fixed speed of 3.2 km/h and an inclination of 12%).

The confirmatory secondary endpoints were a change in maximum walking distance from baseline to week 57, a change in Vascular Quality of Life Questionnaire-6 (VascuQoL-6) score from baseline to week 52 and a change in pain-free walking distance from baseline to week 52.

Supportive secondary endpoints included the physical functioning domain of the Short Form (36) Health Survey change from baseline to week 52, change in ankle-brachial index from baseline to week 52, change in body weight from baseline to week 52 and/or change in glycosylated haemoglobin A1c from baseline to week 52.

A total of 792 patients were randomly assigned 1:1 to receive either 1 mg semaglutide once weekly for 52 weeks or placebo once weekly for 52 weeks. Following end of treatment, outcomes were monitored for 5 additional weeks.

Additionally, all patients received standard of care treatment.

STRIDE included a population of patients with type 2 diabetes and stable symptomatic peripheral arterial disease with intermittent claudication corresponding to Fontaine stage Ila (Rutherford classification grade I, category 1 and 2), with an ankle brachial-index (ABI) s 0.90 or a toe-brachial index (TBI) s 0.70 and having a maximum walking distance (MWD)<600 m on a graded treadmill test.

### Key Inclusion Criteria

Trial participants were male or female, aged 18 years or above or, in Japan and Taiwan, aged 20 years or above with:
Type 2 diabetes mellitus 180 days prior to the day of screening.
Symptomatic PAD with intermittent claudication corresponding to Fontaine stage IIa (Rutherford classification grade I, category 1 and 2), meeting all of the following:
a. Stable symptoms of PAD with intermittent claudication in Fontaine stage Ila (i.e., able to walk without stopping >200 m, 656 feet, or 2 blocks) for 90 days prior to the day of screening.
b. Screening flat treadmill test (3.2 km/h (2 mph)): pain-free walking distance of 200 meters (656 feet).
c. Screening constant load treadmill test with fixed inclination of 12% and a fixed speed of 3.2 km/h (2 mph): walking distance s 600 meters (or 1968 feet).
d. Ankle-brachial-index (ABI) s 0.90 or toe-brachial index (TBI) s 0.70. The leg with the lowest index was chosen in the case of bilateral disease.

### Key Exclusion Criteria

Adults with any one of the following could not participate in the trial:
Current or previous treatment with any GLP-1 receptor agonist within 90 days prior to the day of screening.
Walking ability limited by conditions other than PAD.
Planned orthopaedic surgery in the legs, or other major surgery affecting walking ability.
Vascular revascularisation procedure of any kind 180 days prior to the day of screening.
Planned arterial revascularisation.
Myocardial infarction, stroke, hospitalisation for unstable angina pectoris or transient ischemic attack within 180 days prior to the day of screening.
Heart failure, classified as being in New York Heart Association (NYHA) class III-IV.

### Baseline Characteristics

Trial participants had the baseline characteristics presented in Table 3.

TABLE 3
Baseline Characteristics
Data represented as n (%), mean (standard deviation (SD)),
median (interquartile range (IQR)) or geometric mean (CV)

| Characteristic | Semaglutide | | Placebo | | Total | |
|---|---|---|---|---|---|---|
| Number of subjects | 396 | | 396 | | 792 | |
| Age (years) | 66.2 | (9.71) | 67.0 | (8.96) | 66.6 | (9.35) |

Sex

| | | | | | |
|---|---|---|---|---|---|
| Female | 107 | (27%) | 88 | (22%) | 195 |
| Male | 289 | (73%) | 308 | (78%) | 597 |

Race

| American Indian or Alaska Native | 0 | | 1 | (0.3) | 1 | (0.1) |
|---|---|---|---|---|---|---|
| Asian | 131 | (33.1) | 109 | (27.5) | 240 | (30.3) |
| Black or African American | 4 | (1.0) | 4 | (1.0) | 8 | (1.0) |
| Native Hawaiian or Other Pacific | 1 | (0.3) | 0 | | 1 | (0.1) |
| Islander | | | | | | |
| White | 259 | (65.4) | 279 | (70.5) | 538 | (67.9) |
| Other | 1 | (0.3) | 3 | (0.8) | 4 | (0.5) |

Ethnicity

| | | | | | | |
|---|---|---|---|---|---|---|
| Hispanic or Latino | 7 | (1.8) | 6 | (1.5) | 13 | (1.6) |
| Not Hispanic or Latino | 388 | (98.0) | 385 | (97.2) | 773 | (97.6) |
| Not Reported | 1 | (0.3) | 5 | (1.3) | 6 | (0.8) |
| Duration of diabetes (years) | 12.8 | (8.47) | 13.7 | (9.08) | 13.3 | (8.79) |
| Weight (kg) | 83.39 | (19.28) | 85.05 | (19.10) | 84.22 | (19.19) |
| BMI (kg/m²) | 29.60 | (5.77) | 29.51 | (5.38) | 29.55 | (5.58) |
| Median | 28.6 | (25.6-32.9) | 28.5 | (25.7-33.1) | | |
| <30 | 231 | (58%) | 238 | (60%) | | |

BMI group (kg/m²)

| | | | | | | |
|---|---|---|---|---|---|---|
| ≥25 to <30 | 147 | (37.1) | 157 | (39.6) | 304 | (38.4) |
| ≥30 to <35 | 103 | (26.0) | 94 | (23.7) | 197 | (24.9) |
| ≥35 to <40 | 41 | (10.4) | 49 | (12.4) | 90 | (11.4) |
| ≥40 | 21 | (5.3) | 15 | (3.8) | 36 | (4.5) |
| Systolic BP (mmHg) | 134 | (15.6) | 134 | (16.5) | 134 | (16.1) |
| Diastolic BP (mmHg) | 75 | (9.8) | 74 | (10.0) | 75 | (9.9) |
| HbA_{1c} (%) | 7.2 | (1.03) | 7.4 | (1.12) | 7.3 | (1.08) |
| eGFR (mL/min/1.73 m²) | 84.2 | (19.36) | 82.5 | (20.41) | 83.3 | (19.90) |
| LDL (mmol/L)* | 1.8 | (0.5) | 1.8 | (0.5) | 1.8 | (0.5) |
| LDL (mg/dL)* | 69.2 | (0.5) | 68.7 | (0.5) | 69.0 | (0.5) |

Smoking Status

| | | | | | | |
|---|---|---|---|---|---|---|
| Current | 96 | (24.2) | 107 | (27.0) | 203 | (25.6) |
| Never | 122 | (30.8) | 100 | (25.3) | 222 | (28.0) |
| Previous | 178 | (44.9) | 189 | (47.7) | 367 | (46.3) |

Cardiovascular conditions before screening

| | | | | | | |
|---|---|---|---|---|---|---|
| Coronary heart disease | 162 | (40.9) | 178 | (44.9) | 340 | (42.9) |
| Myocardial infarction | 59 | (14.9) | 89 | (22.5) | 148 | (18.7) |
| Coronary artery stenosis (≥50%) | 106 | (26.8) | 114 | (28.8) | 220 | (27.8) |
| Coronary revascularization | 122 | (30.8) | 129 | (32.6) | 251 | (31.7) |
| Percutaneous coronary intervention | 81 | (20.5) | 94 | (23.7) | 175 | (22.1) |
| Coronary artery bypass graft | 42 | (10.6) | 42 | (10.6) | 84 | (10.6) |
| Stroke | 21 | (5.3) | 30 | (7.6) | 51 | (6.4) |
| Transient ischemic attack | 17 | (4.3) | 14 | (3.5) | 31 | (3.9) |
| Hypertension | 340 | (85.9) | 357 | (90.2) | 697 | (88.0) |
| Chronic kidney disease | 57 | (14.4) | 68 | (17.2) | 125 | (15.8) |
| eGFR <60 mL/min/1.73 m² | 39 | (9.8) | 46 | (11.6) | 85 | (10.7) |
| Chronic heart failure | 55 | (13.9) | 54 | (13.6) | 109 | (13.8) |
| Heart failure with reduced ejection | 15 | (3.8) | 15 | (3.8) | 30 | (3.8) |
| fraction | | | | | | |
| Heart failure with preserved ejection | 34 | (8.6) | 30 | (7.6) | 64 | (8.1) |
| fraction | | | | | | |
| Unknown | 6 | (1.5) | 9 | (2.3) | 15 | (1.9) |

NYHA class

| | | | | | | |
|---|---|---|---|---|---|---|
| I | 22 | (5.6) | 23 | (5.8) | 45 | (5.7) |
| II | 32 | (8.1) | 31 | (7.8) | 63 | (8.0) |

Concomitant Medications at randomization

| | | | | | | |
|---|---|---|---|---|---|---|
| On any concomitant medications at | 394 | (99.5) | 396 | (100) | 790 | (99.7) |
| randomization | | | | | | |

Glucose lowering medication

| | | | | | | |
|---|---|---|---|---|---|---|
| Metformin | 315 | (79.5) | 319 | (80.6) | 634 | (80.1) |
| SGLT2 inhibitors | 146 | (36.9) | 132 | (33.3) | 278 | (35.1) |
| Long acting insulin | 90 | (22.7) | 98 | (24.7) | 188 | (23.7) |
| Short acting insulin | 55 | (13.9) | 77 | (19.4) | 132 | (16.7) |
| Premix insulin | 21 | (5.3) | 22 | (5.6) | 43 | (5.4) |
| Sulphonylureas | 87 | (22.0) | 87 | (22.0) | 174 | (22.0) |
| DPP-4 inhibitors | 80 | (20.2) | 64 | (16.2) | 144 | (18.2) |
| α-Glucosidase inhibitors | 24 | (6.1) | 22 | (5.6) | 46 | (5.8) |
| Pioglitazone | 26 | (6.6) | 12 | (3.0) | 38 | (4.8) |
| Other glucose lowering medication | 6 | (1.5) | 2 | (0.5) | 8 | (1.0) |

Lipid modifying agents

| | | | | | | |
|---|---|---|---|---|---|---|
| Statins | 329 | (83.1) | 324 | (81.8) | 653 | (82.4) |
| Ezetimibe | 58 | (14.6) | 57 | (14.4) | 115 | (14.5) |
| PCSK-9 inhibitors | 5 | (1.3) | 2 | (0.5) | 7 | (0.9) |
| Other lipid modifying drugs | 8 | (2.0) | 8 | (2.0) | 16 | (2.0) |
| Anticoagulant medication | 60 | (15%) | 58 | (15%) | | |
| Direct oral anticoagulants | 50 | (12.6) | 49 | (12.4) | 99 | (12.5) |
| Vitamin K antagonist | 7 | (1.8) | 8 | (2.0) | 15 | (1.9) |
| Other anticoagulant medication | 4 | (1.0) | 1 | (0.3) | 5 | (0.6) |
| (heparin) | | | | | | |
| Antiplatelet medication (Platelet | 255 | (64%) | 261 | (66%) | | |
| aggregation inhibitors) | | | | | | |
| Acetylsalicylic acid | 223 | (56.3) | 226 | (57.1) | 449 | (56.7) |
| P2Y₁₂ inhibitors | 85 | (21.5) | 87 | (22.0) | 172 | (21.7) |
| Cilostazol | 43 | (10.9) | 43 | (10.9) | 86 | (10.9) |
| Other antiplatelet medication | 7 | (0.02) | 13 | (0.03) | 20 | (0.03) |

Other cardiovascular-related medication

| | | | | | | |
|---|---|---|---|---|---|---|
| ACE inhibitors/ARB/ARNI | 291 | (73.5) | 295 | (74.5) | 586 | (74.0) |
| Beta blockers | 190 | (48.0) | 197 | (49.7) | 387 | (48.9) |
| Calcium channel blocker | 152 | (38.4) | 174 | (43.9) | 326 | (41.2) |
| Diuretic | 141 | (35.6) | 158 | (39.9) | 299 | (37.8) |
| Mineralocorticoid receptor | 31 | (7.8) | 28 | (7.1) | 59 | (7.4) |
| antagonists | | | | | | |

TABLE 3b
Baseline Functional Characteristics
Data represented as n, mean (standard deviation (SD)),
median (interquartile range (IQR)) or geometric mean (CV)

| | Semaglutide | Placebo |
|---|---|---|
| Number of subjects | 396 | 396 |

Functional characteristics
Ankle-brachial index

| | | | | |
|---|---|---|---|---|
| Geometric mean | 0.76 | (0.4) | 0.75 | (0.3) |
| Median | 0.79 | (0.64-0.95) | 0.76 | (0.64-0.88) |

Toe-brachial index

| | | | | |
|---|---|---|---|---|
| Geometric mean | 0.48 | (0.4) | 0.48 | (0.4) |
| Median | 0.53 | (0.39-0.64) | 0.50 | (0.38-0.65) |

Maximum walking distance, m

| | | | | |
|---|---|---|---|---|
| Median | 184.5 | (126.5-274.0) | 185.8 | (133.8-262.0) |
| Geometric mean | 192.0 | (0.6) | 188.9 | (0.6) |

Pain-free walking distance, m

| | | | | |
|---|---|---|---|---|
| Median | 119.0 | (76.0-173.5) | 109.0 | (77.8-169.5) |
| Geometric mean | 117.1 | (0.7) | 115.3 | (0.6) |

VascuQoL-6 total score

| | | | | |
|---|---|---|---|---|
| Median | 15.0 | (6.0-24.0) | 15.0 | (7.0-24.0) |
| Mean | 15.2 | (3.8) | 15.1 | (3.6) |

### Treatment Groups and Duration

The total duration of trial participation for each patient was approximately 59 weeks, including a screening period of approximately 2 weeks, a treatment period of 52 weeks and a follow-up period of 5 weeks.

Patients were administered semaglutide (1.34 mg/mL) or placebo. The dosage form was a solution for subcutaneous injection in a 1.5 mL pre-filled PDS290 pen-injector. Semaglutide was administered as a solution having a pH of about 7.4. Semaglutide was administered once weekly in a dose-escalating manner, unless subjects experienced side-effects such as nausea. Most trial participants were treated with 0.25 mg semaglutide from week 1 to week 4, 0.5 mg semaglutide from week 5 to week 8 and 1.0 semaglutide from week 9 to week 52. However, about 3.4% of trial participants remained on the lowest dose of 0.25 mg from week 9 to week 52 and about 10.7% of trial participants received a dose of 0.5 mg from week 9 to week 52. About 86% completed the trial on a dose of 1.0 mg from week 9 to week 52. From week 53-57, neither semaglutide nor placebo was administered.

Functional testing was carried out at baseline (week 0), week 26, week 52 and week 57.

The quantitative effect of once-weekly semaglutide on intermittent claudication was assessed by measuring the maximum walking distance at weeks 52 and 57 and pain-free walking distance at week 52.

A disease-specific patient reported outcomes questionnaire, “Vascular Quality of Life 6 items (VascuQoL-6)”, was used to evaluate patients' experiences of symptoms and function.

A generic patient reported outcomes questionnaire, the “Short Form 36 (SF-36)”, was used to evaluate patients' health-related quality of life.

### Methods

### Treadmill Assessments

Treadmill assessments were only performed on treadmills intended for medical use.

A constant-load treadmill assessment was performed at rate of 2 mph (3.2 km/h) and an inclination of 12%. The results of the constant-load test were expressed in units of distance (m) walked.

Patients were encouraged to walk on the treadmill for as long as possible, disregarding any pain felt. Treadmill testing was discontinued upon the patient's request, or in the absence of claudication, after a total walking duration of 20 minutes. The patient was to indicate when pain started to be felt in either leg (pain-free walking distance) and then continue walking on the treadmill without stopping, until it was impossible for him or her to walk any further. This was the “maximum walking distance”.

The baseline treadmill assessment was performed at the beginning of the baseline visit (week 0) and at the end of the baseline visit, to evaluate variability in the single patient's performance. As a minimum, the two assessments were one hour apart. A duplicate assessment was only needed at baseline.

### Pain-Free Walking Distance on an Inclined Treadmill

The pain-free distance walked on the inclined treadmill (having a fixed speed of 3.2 km/h (2 mph)) and a fixed inclination of 12%) was measured at the screening (week −2), baseline (week 0), week 26, end of treatment (week 52) and follow-up (week 57) visits.

### Maximum Walking Distance on an Inclined Treadmill

The maximum distance walked on the inclined treadmill (having a fixed speed of 3.2 km/h (2 mph)) and a fixed inclination of 12%) was measured at the screening (week −2), baseline (time 0), week 26, end of treatment (week 52), and follow-up (week 57) visits.

Upon screening, participants had to demonstrate a maximum walking distance of less than 600 m. This criterion was applied to reduce heterogeneity of the trial population.

Maximum walking distance at week 57 (5 weeks after semaglutide/placebo discontinuation) was measured to evaluate persistence of effect.

### Pain-Free Walking Distance on a Flat Treadmill

Proper classification of trial participants' Fontaine Ila claudication needed to be confirmed on a flat treadmill, mimicking a normal walking situation, at the screening visit. If the patient was able to walk at least 200 m (656 feet) on a flat treadmill with a fixed speed of 3.2 km/h (2 mph) before onset of pain in either leg, the patient was potentially eligible to participate in the trial.

### Ankle-Brachial Index (ABI) and Toe-Brachial Index (TBI)

The ankle-brachial index (ABI) and toe-brachial index (TBI) measurements were performed in the supine patient after at least 5 minutes' rest. The measurements were performed by an automatic calibrated Sphygmomanometer or using the Doppler method. If both methods were available, the Doppler method was preferred. The same method was used throughout the trial for each individual patient. The brachial blood pressure was measured at the same time as each toe or ankle pressure. ABI and TBI were calculated averages of two measurements in each location: at each scheduled assessment, two measurements were performed at each location (i.e., both right and left ankle, arm and toe).

The ABI was calculated as the ratio of the higher ankle systolic pressure to the higher systolic pressure measured in both arms. The ankle pressure was preferably measured using a Doppler probe to obtain systolic pressures in the following locations: posterior tibial and anterior tibial artery systolic pressures. The higher of the tibial pressure was chosen for the numerator for each leg, and the higher of the left and right brachial systolic pressure was chosen for the denominator. The ankle pressure was measured using a blood pressure cuff chosen according to the limb size. The width of the cuff needed to contour at least 40% of the limb circumference. If a Doppler device was used, an 8-10 MHz Doppler probe was used (standard vascular probe).

The TBI was calculated as a ratio of the toe systolic pressure to the higher systolic pressure measured in both arms. If toe pressure was measured by laser Doppler flowmetry (LDF), this was with linear deflation pressure and a cuff that was at least 1.2 times wider than the toe (usually a 2.5-3.0 cm cuff for the great toe). The Doppler probe was applied to the plantar surface of the distal portion of the great toe (or in absence of it on the second toe), and the occlusive cuff was placed around the proximal portion of the toe.

The ABI and TBI were measured and noted for both legs and toes throughout the trial.

### Vascular Quality of Life 6 Items (VascuQoL-6)

A more comprehensive overall PAD-related Quality of Life and functional measurement was performed using the VascuQoL-6 questionnaire (Nordanstig et al., Vascular Quality of Life Questionnaire-6 facilitates health-related quality of life assessment in peripheral arterial disease. J Vasc Surg 2014; 59:700e7). The questionnaire comprised 6 items covering 5 domains: social, emotional, functional, pain- and symptom-related aspects of the patient's overall quality of life. Each item was scored on a four-point response scale (1=worst; 4=best), with higher scores indicating a better health status. The total score ranged from 6 to 24 points.

### Short Form 36

Overall health-related quality of life was evaluated using the Short Form 36 v2.0 acute (SF-36) (Ware J E, Jr., Sherbourne C D. The MOS 36-item short-form health survey (SF-36). I. Conceptual framework and item selection. Med Care. 1992; 30(6):473-83. SF-36 is a generic measure of health status that yields 2 summary scores for physical health and mental health and 8 domain scores. (Ware J E, Jr., Sherbourne C D. The MOS 36-item short-form health survey (SF-36). I. Conceptual framework and item selection. Med Care. 1992; 30(6):473-83.)

### Results

The effect of semaglutide 1.0 mg was found to be superior to placebo from baseline to both week 52 and week 57 with regards to all confirmatory tests:
Maximum Walking Distance (ratio) at week 52 (see Tables 4 and 7).
Maximum Walking Distance (ratio) at week 57 (see Tables 5 and 7).
VascuQoL-6 total score (change) at week 52.
Pain-Free Walking Distance (ratio) at week 52 (see Tables 6 and 7).

TABLE 4
Maximum walking distance at week 52

| | | |
|---|---|---|
| Maximum walking | Semaglutide | Placebo |

| | |
|---|---|
| distance (m) | Median (Interquartile range (IQR)) |

| Baseline | 184.50 (147.50) | 185.75 (128.25) | |
|---|---|---|---|
| Week 52 | 223.50 (228.00) | 214.00 (173.00) | |
| Ratio to baseline | 1.21 (IQR: 1.0-1.6 = | 1.08 (IQR: 0.9-1.4 = | |
| at week 52 | 0.60) | 0.50) | |
| | Hodges - Lehmann | | |
| | estimate of location | | |
| Analysis at week 52 | shift | 95% CI | p-value |
| Semaglutide/Placebo | 1.13 | [1.056; 1.211] | 0.0004 |

At week 52, the estimated median ratio to baseline in maximum walking distance was significantly greater in the semaglutide group than in the placebo group (see Table 4). At week 52, the observed median ratio to baseline of maximum walking distance was greater in the semaglutide group than in the placebo group.

The improvement at week 52 in maximum walking distance was significantly greater in the semaglutide treatment group than in the placebo treatment group (see Tables 4 and 7).

At week 57, five weeks after treatment cessation, the improvement in maximum walking difference remained significantly greater in the semaglutide treatment group than in the placebo treatment group (see Tables 5 and 7).

TABLE 5
Maximum walking distance at week 57

| Maximum walking | Semaglutide | Placebo |
|---|---|---|
| distance (m) | Median (Interquartile Range (IQR)) | |
| Baseline | 184.50 (147.50) | 185.75 (128.25) |
| Week 57 | 213.00 (215.00) | 212.00 (172.00) |
| Ratio to baseline | 1.16 (IQR: 0.9-1.5 = 0.6) | 1.10 (IQR: 0.9-1.4 = 0.5) |
| at week 57 | | |

| | Hodges - Lehmann | | |
|---|---|---|---|
| | estimate of location | | |
| Analysis at week 57 | shift | 95% CI | p-value |
| Semaglutide/Placebo | 1.08 | [1.004; 1.156] | 0.0380 |

The improvement at week 52 in pain-free walking distance was significantly greater in the semaglutide treatment group than in the placebo treatment group (see Table 6).

TABLE 6
Pain-free walking distance at week 52

| Pain-free walking | Semaglutide | Placebo |
|---|---|---|
| distance (m) | Median (Interquartile Range (IQR)) | |
| Baseline | 119.00 (97.50) | 109.00 (91.75) |
| Week 52 | 142.00 (129.00) | 123.50 (125.50) |
| Ratio to baseline | 1.21 (IQR: 0.9-1.5 = 0.60) | 1.10 (0.9-1.4) |
| at week 52 | | |

| | Hodges - Lehmann | | |
|---|---|---|---|
| | estimate of location | | |
| Analysis at week 52 | shift | 95% CI | p-value |
| Semaglutide/Placebo | 1.11 | [1.033; 1.197] | 0.0046 |

TABLE 7
Functional Outcomes

| | | Estimated | | |
|---|---|---|---|---|
| | Analysis | Treatment | | p- |
| Endpoint | variable | Ratio | 95% CI | value |
| Change in Maximum | Semaglutide/ | 1.14 | [1.058; | 0.0005 |
| walking distance at | Placebo | | 1.219] | |
| week 52 | | | | |
| Change in Maximum | Semaglutide/ | 1.07 | [1.001; | 0.0462 |
| walking distance at | Placebo | | 1.153] | |
| week 57 | | | | |
| Change in Pain-free | Semaglutide/ | 1.15 | [1.061; | 0.0007 |
| walking distance at | Placebo | | 1.244] | |
| week 52 | | | | |

Furthermore, the ankle-brachial index significantly increased in the population that received semaglutide (see Table 8).

TABLE 8
Supportive end-points at week 52
Data represented as a mean (standard
deviation (SD)) or geometric mean (CV)

| Analysis at week 52 | Semaglutide | | Placebo | | p-value |
|---|---|---|---|---|---|
| Geometric mean ankle- | 1.06 | (34.0) | 1.02 | (19.6) | 0.0037 |
| brachial index, ratio to | | | | | |
| baseline | | | | | |
| Mean change from baseline | 5.2 | (4.8) | −1.2 | (4.2) | <0.0001 |
| in bodyweight, kg | | | | | |
| Mean change from baseline | −0.8% | (1.1) | 0.2% | (1.1) | <0.0001 |
| in HbA1c | | | | | |

In the exploratory analysis, the difference between the groups in the median absolute improvement in pain-free walking distance was 13.8 m (95% Cl 4.1-23.5) and the difference in the mean absolute improvement was 29.8 m (SD: 11.6-48.0).

TABLE 9
Exploratory end-points at week 52
Data represented as a mean (standard deviation
(SD)) or median (interquartile range (IQR))

| Analysis at week 52 | Semaglutide | | Placebo | |
|---|---|---|---|---|
| Median absolute improvement | 37 | (−8.0 to 109.0) | 13 | (−26.5 to 70.0) |
| in maximum walking | | | | |
| distance, m | | | | |
| Mean absolute improvement | 87 | (182) | 41 | (152) |
| in maximum walking | | | | |
| distance, m | | | | |
| Median absolute improvement | 22 | (−9.0 to 65.5) | 12 | (−14.0 to 52.8) |
| in pain-free walking | | | | |
| distance, m | | | | |
| Mean absolute improvement | 52 | (124) | 23 | (106) |
| in pain-free walking | | | | |
| distance, m | | | | |

### SF-36 Physical Functioning

At baseline (week 0), the observed mean (SD) SF-36 Physical Functioning domain score was 42.13 (7.39) for participants in the semaglutide treatment group and 42.12 (7.02) for participants in the placebo treatment group.

At week 52, the estimated change (SE) from baseline (week 0) in SF-36 Physical Functioning domain score was 2.97 (0.358) for participants in the semaglutide treatment group and 1.72 (0.352) for participants in the placebo treatment group.

The improvement in the physical functioning domain of the SF-36 was significantly greater in the semaglutide treatment group versus the placebo treatment group (estimated treatment difference 1.25 [95% Cl 0.26-2.24]; p=0.013).

### Further Findings

Participants in both treatment groups (semaglutide/placebo) lost weight between baseline and week 52: there was a mean change of −5.2 kg (SD: 4.8) in the semaglutide group and −1.2 kg (SD: 4.2) in the placebo group (estimated treatment difference −4.1 (95% Cl −4.8 to −3.4; p<0.0001). The association between the change in BMI and the primary endpoint of maximum walking distance at week 52 was assessed in each treatment group. There was a statistically significant association between BMI change and function in the placebo group; however, the relationship as measured by Spearman's R was weak (−0.1410; p=0.014). Similarly, there was a weak relationship in the semaglutide group (Spearman's R −0.1260; p=0.031; appendix p 45).

The BMI of the trial population as a whole (median BMI 28.6) was typical of populations with peripheral artery disease, who tend not to have obesity. This tendency, coupled with the modest observed weight loss of a mean of 5.2 kg in trial participants that received semaglutide, the improvements in pain-free walking distance and ankle-brachial index, suggests a direct vascular benefit of semaglutide rather than benefits mediated solely by weight loss.

### Conclusions

STRIDE met its primary and key secondary objectives by demonstrating a favorable effect of semaglutide s.c. 1.0 mg on both walking ability and patient-reported symptoms and impacts of intermittent claudication (VascuQoL-6), compared to placebo.

Superiority of semaglutide vs placebo was confirmed for maximum walking distance at week 52. Patients' maximum walking distance was improved by 13%.

Superiority of semaglutide vs placebo was confirmed for maximum walking distance at week 57. Patients' maximum walking distance remained improved at week 57; that is, five weeks following the discontinuation of treatment. An improvement in maximum walking distance at week 57 demonstrated that the improvement seen after 52 weeks' treatment was potentially due to a change in pathophysiology and not only due to symptomatic relief.

Superiority of semaglutide vs placebo was confirmed for VascuQoL-6 total score at week 52 (1-point median improvement).

Superiority of semaglutide vs placebo was confirmed for pain-free walking distance at week 52. Patients' pain-free walking distance was improved by improved by 11%.

Overall, semaglutide improved function, symptoms and quality of life in people with symptomatic peripheral artery disease and type 2 diabetes. Semaglutide significantly improved maximum walking distance. Furthermore, there were statistically significant improvements in all confirmatory secondary endpoints, including pain-free walking distance (symptoms) and quality of life.

Surprisingly, semaglutide appears to have a direct vascular effect on peripheral arteries that are partially occluded by atherosclerosis, suggested by the improvement in ankle-brachial index and manifesting as an improvement in the symptoms of claudication. Semaglutide thus contributes to reversing an otherwise slowly progressive disease, peripheral arterial disease.

1. A method for the improvement of intermittent claudication in a subject in need thereof, comprising administering semaglutide to the subject, wherein the subject has type 2 diabetes and peripheral arterial disease (PAD).

2. The method according to claim 1, wherein the subject has intermittent claudication corresponding to Fontaine stage IIA.

3. The method according to claim 1, wherein the subject has an ankle-brachial index (ABI) equal or less than 0.90.

4. The method according to claim 3, wherein the subject has an ABI equal to or more than 0.40.

5. The method according to claim 1, wherein the subject has a toe-brachial index (TBI) equal to or less than 0.70.

6. The method according to claim 1, wherein the subject has an ankle-brachial index (ABI) equal or less than 0.90 and a toe-brachial index (TBI) equal to or less than 0.70.

7. The method according to claim 6, wherein the ABI is equal to or more than 0.40.

8. The method according to claim 1, wherein the treatment increases the maximum distance that the subject is capable of walking.

9. The method according to claim 1, wherein the treatment increases the distance that the subject is capable of walking without feeling pain.

10. The method according to claim 1, wherein the treatment increases the ankle-brachial index (ABI) of the subject.

11. The method according to claim 1, wherein the treatment increases the toe-brachial index (TBI) of the subject.

12. The method according to claim 1, wherein the semaglutide is administered in an amount of 0.2-50 mg.

13. The method according to claim 1, wherein the semaglutide is administered in a liquid formulation.

14. The method according to claim 13, wherein the liquid formulation comprises 0.25-22 mg/ml semaglutide.

15. The method according to claim 13, wherein the semaglutide is administered in an amount of about 0.25 mg.

16. The method according to claim 13, wherein the semaglutide is administered in an amount of about 0.5 mg.

17. The method according to claim 13, wherein the semaglutide is administered in an amount of about 1.0 mg.

18. The method according to claim 13, wherein the semaglutide is administered in an amount of about 1.7 mg.

19. The method according to claim 13, wherein the semaglutide is administered in an amount of about 2.0 mg.

20. The method according to claim 13, wherein the semaglutide is administered in an amount of about 2.4 mg.

21. The method according to claim 13, wherein the semaglutide is administered in an amount of about 7.2 mg.

22. The method according to claim 1, wherein the semaglutide is administered orally in a solid formulation.

23. The method according to claim 22, wherein the semaglutide is administered in an amount of about 25 mg.

24. The method according to claim 22, wherein the semaglutide is administered in an amount of about 50 mg.

25. The method according to claim 1, wherein the semaglutide is administered about once weekly.

26. The method according to claim 25, wherein the semaglutide is administered once weekly.
